(19) United States
(12) Patent Application Publication
(10) Pub. No.: US 2013/0204160 A1
(43) Pub. Date: Aug. 8, 2013

(54) SYSTEM, METHOD AND APPARATUS FOR HARVESTING BONE MARROW

(71) Applicants: Wade McKenna, Decatur, TX (US); Charles Bolles, Ocala, FL (US); Luke Whalen, Ocala, FL (US); Stephen C. Bales, Ocala, FL (US)

(72) Inventors: Wade McKenna, Decatur, TX (US); Charles Bolles, Ocala, FL (US); Luke Whalen, Ocala, FL (US); Stephen C. Bales, Ocala, FL (US)

(73) Assignee: Biologic Therapies, Inc., Ocala, FL (US)

(21) Appl. No.: 13/838,205

(22) Filed: Mar. 15, 2013

Related U.S. Application Data
(63) Continuation-in-part of application No. 13/403,957, filed on Feb. 23, 2012.
(60) Provisional application No. 61/445,624, filed on Feb. 23, 2011.

(51) Int. Cl. A61B 10/02

(52) U.S. Cl. CPC A61B 10/025
USPC 600/566; 600/567

(57) ABSTRACT

Embodiments of a system, method and apparatus for harvesting bone marrow are disclosed.

### CROSS REFERENCE TO RELATED APPLICATIONS

This application is a continuation-in-part of U.S. patent application Ser. No. 13/403,957, filed on Feb. 23, 2012, entitled, System And Method To Harvest Bone Marrow, which claims the benefit of U.S. Provisional Application No. 61/445,624, filed on Feb. 23, 2011, entitled, Process To Create Chondrogenesis, the disclosures of which are incorporated herein by reference for all purposes.

### TECHNICAL FIELD

The invention relates in general to cell harvesting techniques, and in particular to kits, processes and methods for harvesting bone marrow.

### BACKGROUND INFORMATION

As is well known, articular cartilage is a smooth resistant elastic tissue covering the terminal part of a bone at major joints within human and animal bodies. Such cartilage facilitates movement of the joint and absorbs shock. Through age, disease, or trauma, this cartilage often becomes damaged causing osteochondral defects. When, there is an inadequate amount of this cartilage or an osteochondral defect, pain and swelling often occurs. Once damaged, the cartilage has limited repair capabilities.

Mesenchymal stem cells (MSC) are pluripotent blast or embryonic-like cells located in blood, bone marrow, dermis, adipose tissue and perisosteum. In general these cells are capable of renewing themselves over extended periods of time as well as, under various environmental conditions, differentiating into cartilage, bone and other connective tissue. Recently, various investigators have researched the potential for using these cells to repair or regenerate target tissues, e.g., bone, cartilage, etc. in this manner MSCs have been reported to have regenerative capabilities in a number of animal models.

During standard procedures to isolate bone marrow, a standard Jamshidi® bone marrow biopsy needle or other needles that leave a large diameter hole are typically used. Since there is only a point of entry at the tip of the needle; a large vacuum is generated during the aspiration process resulting in potentially a large amount of cell lysis during extraction and/or other complications.

While such procedures have been used to harvest bone marrow, they have not been found to show an adequate volume of bone marrow per aspiration or an adequate quality of bone marrow such as cell volume or cell viability. What is needed, therefore, is an apparatus and method of increasing the efficiency of obtaining gentle, unrestricted material flow upon aspiration of bone marrow.

### SUMMARY

In response to these and other problems, in one embodiment, there is an apparatus or “kit” for use in harvesting bone marrow, the kit comprising: a needle having a distal end portion and a proximal end portion, including: a longitudinal bore running from the distal end portion to the proximal end portion, one or more fenestrations defined on a side wall of the distal end portion to allow access from an external portion of the side wall to the longitudinal bore, a trocar having a longitudinal shaft, wherein the longitudinal shaft is sized to be inserted into the needle and has a length such that a distal tip of the longitudinal shaft extends beyond the distal end portion of the needle when the longitudinal shaft is inserted into the needle; and a plunger having a plunger longitudinal shaft, wherein the plunger longitudinal shaft is sized to be inserted into the longitudinal bore and has a plunger shaft length such that a portion of a distal end of the plunger longitudinal shaft extends beyond the distal end portion of the needle when the longitudinal shaft is inserted into the needle.

In other embodiments, there are described methods of efficiently harvesting bone marrow.

These and other features, and advantages, will be more clearly understood from the following detailed description taken in conjunction with the accompanying drawings. It is important to note the drawings are not intended to represent the only aspect of the invention.

### BRIEF DESCRIPTION OF THE DRAWINGS

FIGS. 1A-1H shows various views of an intracancellous cell extraction needle of the subject invention. FIG. 1A shows an exploded view of the needle assembly. FIG. 1B shows a proximal end view of a needle grip. FIG. 1C shows a 25 mm needle. FIG. 1D shows a 45 mm needle. FIG. 1E shows a 60 mm needle. FIG. 1F shows an 80 mm needle. FIG. 1G shows a 105 mm needle. FIG. 1H shows the distal tip of a needle insert.

FIGS. 2A-2C illustrate an exemplary trocar. FIG. 2A shows an assembled view of a trocar. FIG. 2B shows an exploded view of the trocar and one embodiment of the needle of FIGS. 1A-1H. FIG. 2C shows a proximal end view of a trocar grip.

FIG. 3A shows an assembled needle drill assembly.

FIG. 3B shows an exploded view of the needle drill assembly of FIG. 3A and the coupling to an exemplary handheld drill.

FIG. 3C shows a perspective view of a drive chuck coupler.

FIG. 4 shows a representative assembled embodiment of a plunger.

FIGS. 5A and 5B show embodiments relating to the plunger. FIG. 5A shows an exploded view of the plunger and a needle. FIG. 5B shows an assembled view of the plunger and the needle.

FIGS. 6A-6C show embodiments relating to the aspiration assembly. FIG. 6A shows an exploded view of the suction device and the needle. FIG. 6B shows an assembled view of the suction device and the needle. FIG. 6C shows a tubing assembly that can be used to couple the suction device and the needle.

FIG. 7 shows an illustrative How chart of one process to harvest bone marrow.

FIGS. 8A-8E are detailed conceptual illustrations of a cross section of bone marrow illustrating the exemplary process of FIG. 7.

FIGS. 9A-9B provide representative sites of extraction. FIG. 9A shows the front side or ventral side and FIG. 9B shows the backside or the dorsal side.

FIGS. 10A-10G provide illustrations of an exemplary process to extract bone marrow from a vertebral site.

FIG. 11 is a perspective view of an exemplary kit containing a needle and a plunger.

### DETAILED DESCRIPTION

For the purposes of promoting an understanding of the principles of the present inventions, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the inventions as described herein are contemplated as would normally occur to one skilled in the art to which the invention relates.

Unlike the biopsy needles that have historically been used, the embodiments described herein relate to a kit and method for harvesting bone marrow from cancellous bones or other bones having red bone marrow potential using a fenestrated cannula, which thereby increases the intake area of the device. It is envisaged that a fenestrated catheter can capture more marrow thereby increasing the cell count with one aspirate. Furthermore, the fenestrations may also reduce the pressure on the marrow resulting in a less traumatic event and potentially increasing cell viability, creates multiple access points, decreases the turbidity of the fluid draw, decreases clot formation, and decreases platelet activation which results in lower platelet activation, lower clot formation.

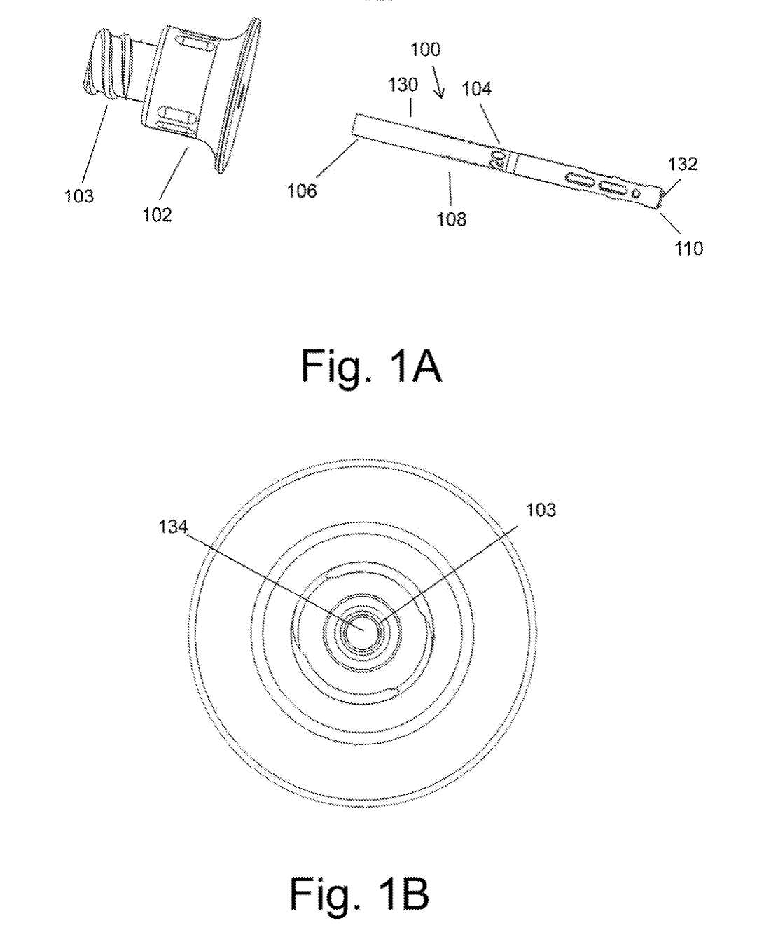

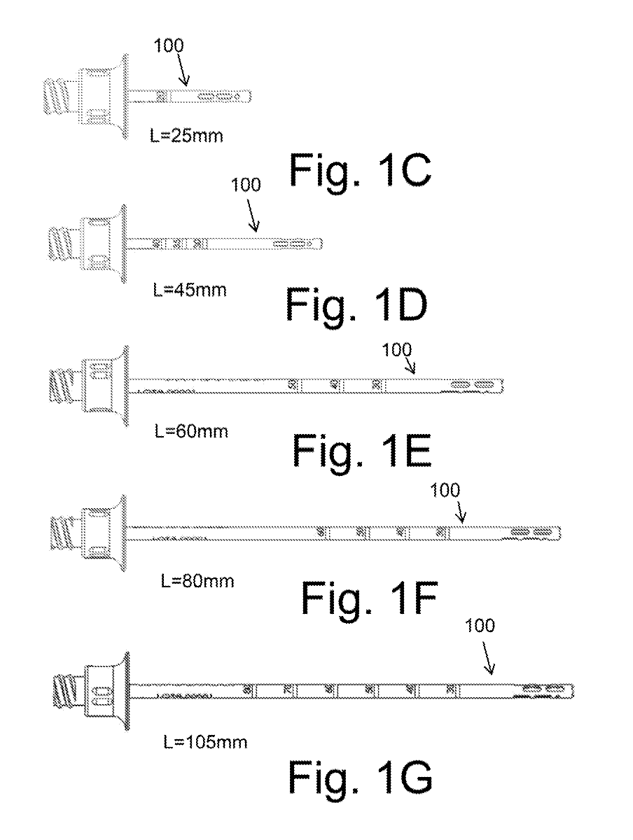

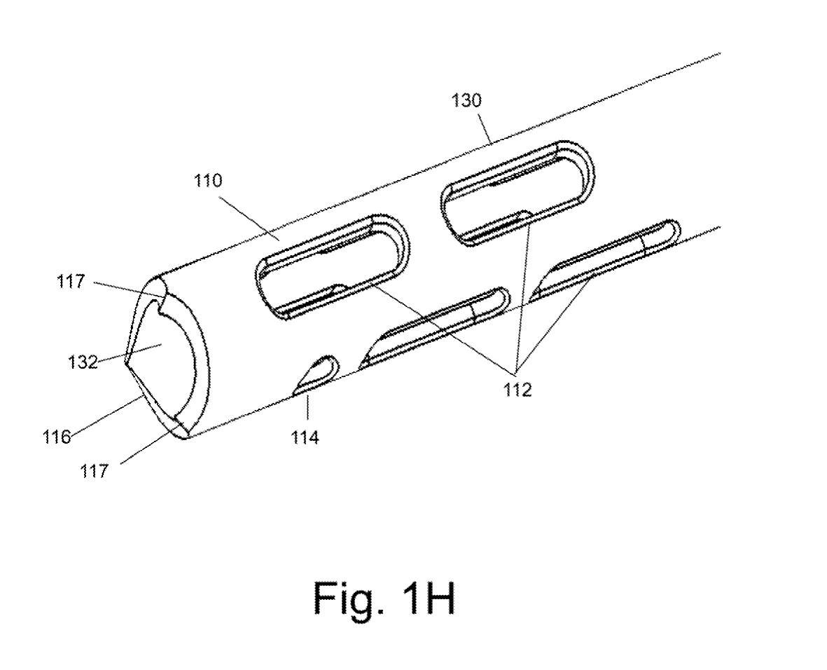

Turning to FIG. 1A, there is illustrated an exploded view of one embodiment of a fenestrated needle 100 that may be used to capture or aspirate bone marrow. Although the term “needle” is used herein, those of skill in the art may also use the terms cannula or sleeve, or other terms which refer to a hollow cylindrical tube. In certain embodiments, the needle 100 may include a needle body or insert 104. In certain embodiments, the needle insert 104 may be constructed of 316LVM stainless steel or a biocompatible corrosion resistant equivalent material, such as 304 stainless steel or nitinol. A needle grip 102 may be coupled to a proximal end 106 of needle insert 102. In certain embodiments, the needle grip 102 may be constructed from polyetherimide material or a biocompatible equivalent such as a polycarbonate equivalent. The needle grip 102 may be overmolded onto the stainless insert 104 via an injection molding process. The needle insert 104 can include an exterior surface 130, a proximal end 108, an intermediate or medial section 103, a distal end (or end portion) 110 and a longitudinal bore 132 that can extend from the proximal end to the distal end.

In certain embodiments, the proximal end 103 of the needle grip 102 has standard thread lengths and helices for compatibility with standard coupling mechanisms, such as leur-locks. FIG. 1B illustrates a proximal or back view of the needle grip 102 showing male threads without are sized and formed for leur-lock compatibility when a trocar or plunger (described below) is coupled to the needle grip 102. A bore 134 runs longitudinally through the needle grip 102. In certain embodiments, there is a slight taper in the bore 134 to enable the fitting or insertion of the various components into the needle 100.

The exterior surface 130 is generally smooth to enable the needle insert 104 to pass through the body. In certain embodiments, both the proximal end 106 and the distal end 110 of the needle insert 104 can have a relatively fixed length while the length of the intermediate or medial section 108 can vary depending upon the site of insertion or the animal in which the needle is used. For example, in certain embodiments, the overall length of the needle can be about 25 mm, 45 mm: 60 mm, 80 mm, as shown in FIGS. 1C, 1D, 1E, 1F respectively. The overall length of the needle can also be 105 mm, as shown in FIG. 1G. The inside and outside diameters of the sleeve 104 and the outside diameter of the needle 200 and plunger 400 can also vary depending upon the insertion site or animal in which the needle is used. The outside diameter of the sleeve 104 can be between 2.4 mm and 3.0 mm, and the inside diameter of the sleeve 104 can range between 1.83 mm and 2.4 mm, with the larger inside diameter only being used when paired with the larger outside diameter. The outside diameters of the needle 200 and plunger 400 can range between 1.53 mm and 2.4 mm, to be compatible with the corresponding inside diameter of the sleeve 104. In other embodiments, the dimensions of the needle 100 can be adjusted such that size of the needle insert 104 can be sufficient to be positioned at a desired depth into the body of the subject while enabling the user to grasp and manipulate the needle. For example, the lengths of needle insert 104 may vary depending upon the extraction site, such as the proximal tibia, spinal vertebra, iliac crest or vary depending upon the mammal in which the extraction may be preformed, for example, other animals may include primates, canines, felines, horses, elephants, dolphins, etc. Depending on such sites and animals, exemplary ranges for the lengths may include about 2.5× greater to about 5× greater than the exemplary lengths of 25 mm, 45 mm, 60 mm, or 80 mm.

FIG. 1H is an expanded view of the distal end portion 110 of needle 100 showing the exemplary fenestrations 112 and 114. In certain embodiments, the fenestrations 112 and 114 can vary in number, size and/or shape. For example, the length of the fenestrations 112 can be, but are not limited to about 2.5 mm in length having a radius of about 0.4 mm. in certain embodiments, the radius of the fenestrations can be altered in the range of about plus or minus five percent. In yet other embodiments, the number of fenestrations can vary depending upon the length of the needle insert 104. In the exemplary embodiment shown in FIG. 1H, oblong, radiused fenestrations 112 may be defined in the wall of the distal end 110. The fenestrations 112 may be separated by a predetermined longitudinal distance, for instance 1.9 mm, with a total of about eight fenestrations. In certain embodiments, there may be more or fewer fenestrations,

Additionally, in other embodiments, the shape of the fenestrations can be altered, for example, the fenestrations may have a circular, oval, polygonal or amorphous shape. In certain embodiments, there may be at least one circular fenestration 114 defined with in the waif of the needle insert 104. In some embodiments, circular fenestration 114 may have a circumference of about 0.8 mm.

Yet further, as illustrated in FIG. 1H, the distal end portion 110 of the needle insert 104 can terminate at a distal tip 116. In certain embodiments, the distal tip 116 may be formed to define a plurality of cutting points 117. The cutting points 117 of the distal tip 116 increases or maximizes the cutting efficiency of the needle insert 104 (as described below) while aiding in the gentle, unrestricted material flow during aspiration.

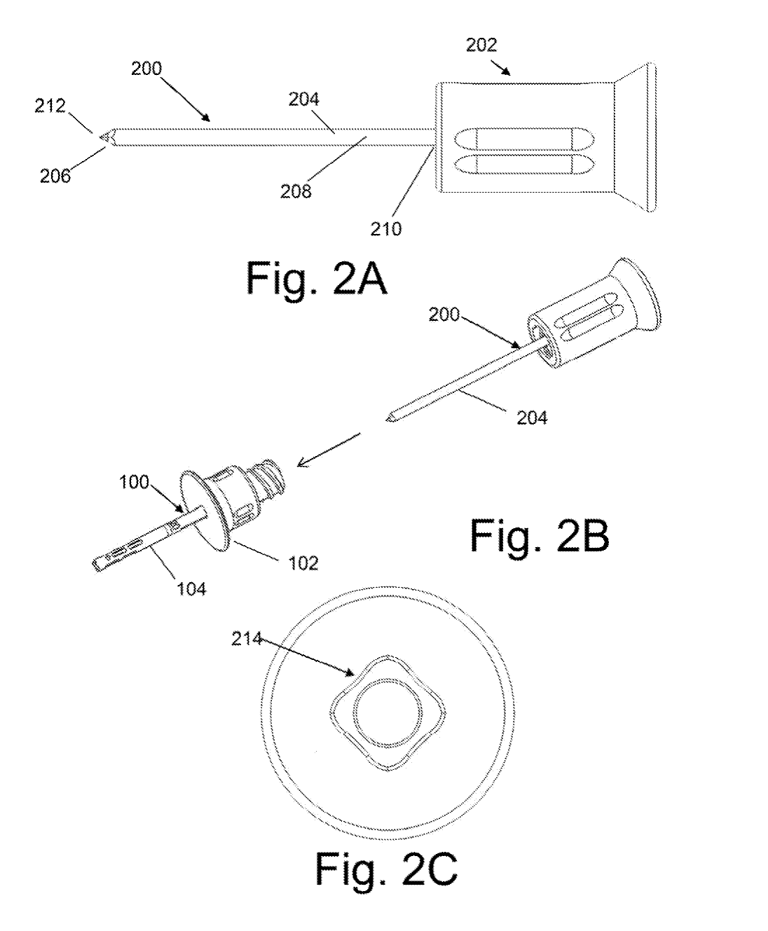

FIG. 2A shows an assembled side view of a solid cylinder shaped trocar 200 to be used in conjunction with needle 100 (FIGS. 1A-1G). The trocar 200 has a trocar grip or cap 202 coupled to the trocar insert 204. The trocar insert 204 has a proximal end 210 (shown seated into grip 202), a distal end (or distal end portion) 206 and an intermediate or medial section 208. In certain embodiments, the trocar insert 204 may be constructed of 318LVM stainless steel or a biocompatible corrosion resistant equivalent such as 304 stainless steel. In certain embodiments, the grip 202 may he constructed from a polyethermide material or a biocompatible equivalent such as a polycarbonate. In certain embodiments, the trocar grip 202 may be overmolded onto the stainless insert 204 via an injection molding process.

The length of the trocar insert 204 may vary depending upon the length of the corresponding needle 100, For example, if needle 100 is 25 mm in length, then the trocar 200 may be about 40.3 mm in length. Thus, in certain embodiments, the length of trocar 200 is about 1.6× the length of the needle 100, The distal end 206 comprises a distal tip 212 having radiused geometry to aide in the cutting efficiency. In certain embodiments, the distal tip 212 has a triangular shape.

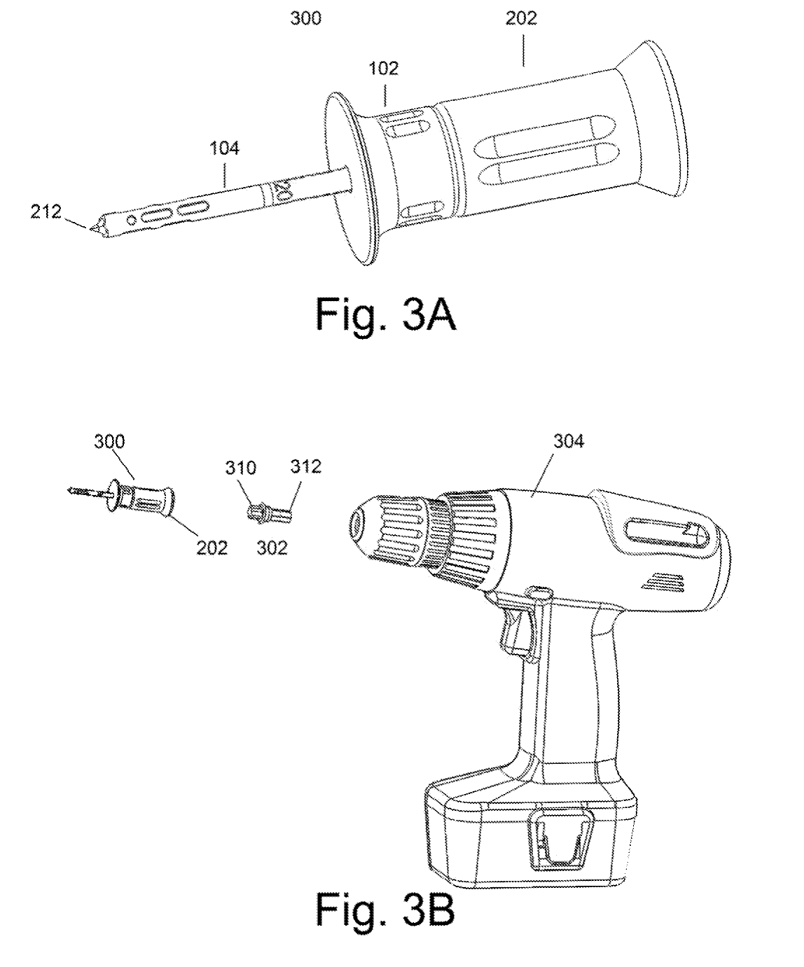

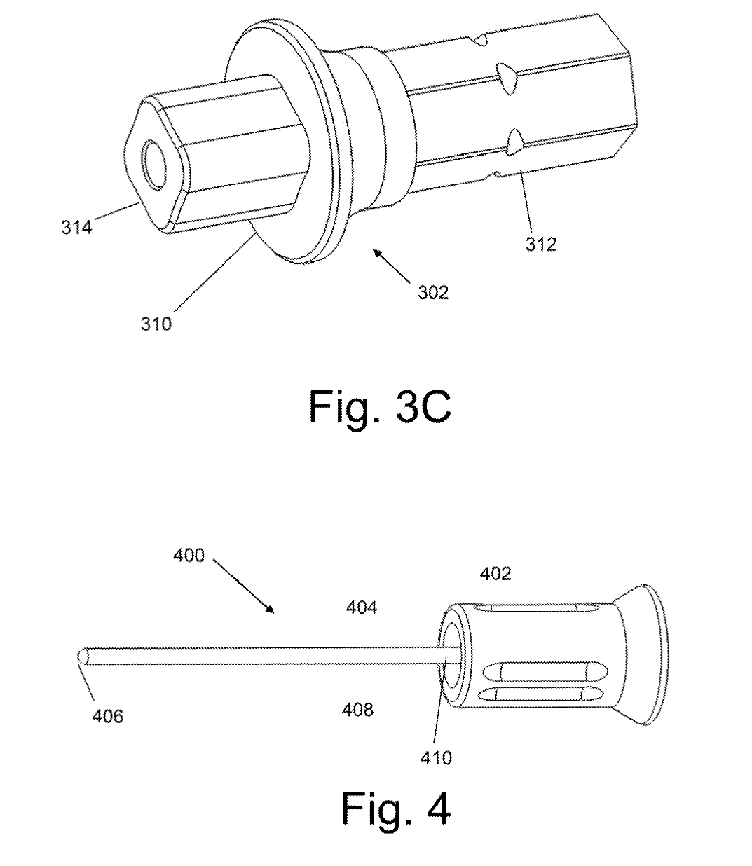

FIG. 2B is an exploded view of the trocar 200 and the needle 100. In certain embodiments, the needle insert 204 may be inserted into the bore 134 in the proximal end of the needle grip 102 and pushed through until the trocar grip 204 contacts the needle grip 102. Then the appropriate threads and grooves may be engaged to lock the trocar grip 204 to the needle grip 104 to form a needle assembly 300, as shown in FIG. 3A. As referred to herein, the “needle assembly” relates to the assembled needle 100 and trocar 200, as shown in FIG. 3A.

FIG. 2C illustrates the back view of the trocar grip 202 showing a socket 214 sized to allow for coupling with a drive chuck coupler 302.

One embodiment of a drive chuck coupler 302 is shown in FIG. 38 and FIG. 3C. In certain embodiments, the drive coupler 302 may be manufactured from 6061-T6 aluminum or an equivalent thereof. In certain embodiments, the drive coupled 302 has a proximal end 312 and a distal end 310 (or end portion) in which a tip 314 projects from the surface of the distal end 310. The shape of the tip 314 is compatible for coupling with the socket 214. In certain embodiments, the proximal end 312 is sized to be inserted into a wide varieties of handheld drills, such as handheld drill 304. The drive mechanism 302 allows the trocar 202 to be used with a wide variety of drills or driving devices typically used in an operating room environment, a clinic facility or an outpatient facility.

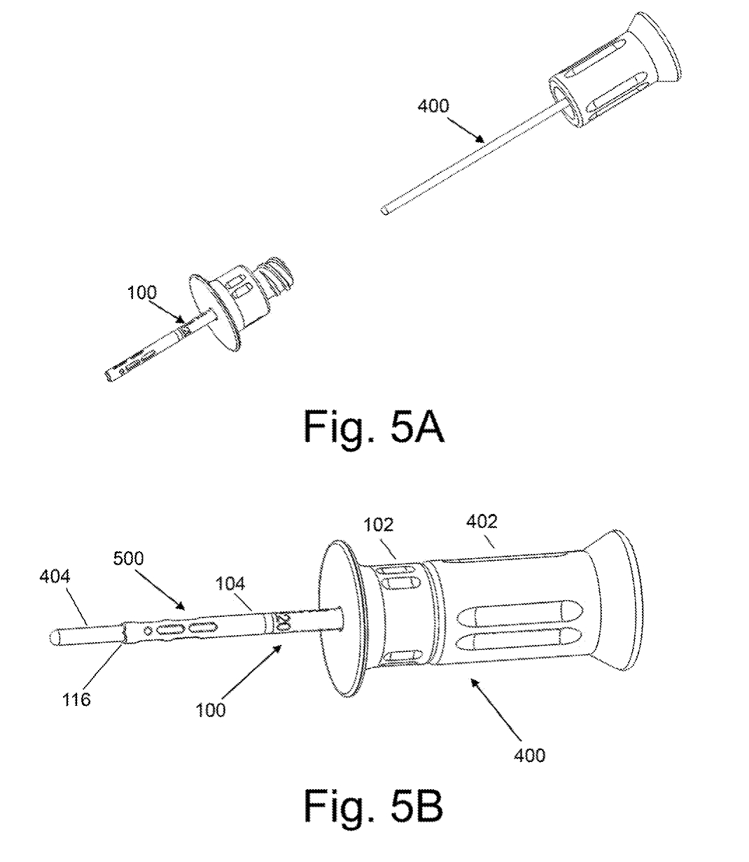

Turning to FIGS. 4 through 5B, there is a plunger 400 that may be manually deployed into the needle 100 after the trocar 200 is removed. FIG. 4 shows a perspective view of the plunger 400, which if contains a grip 402 and a plunger insert 404. In certain embodiments, the plunger insert 404 may be constructed of 318LVM stainless steel or a biocompatible corrosion resistant equivalent, for example, such as 314 stainless steel.

In certain embodiments, the plunger grip 402 is constructed from polyethermide material or a biocompatible equivalent such as a polycarbonate equivalent. In certain embodiments, the plunger grip 402 is overmolded onto the plunger insert 404 via an injection molding process. In certain embodiments, the plunger 400 has a proximal end 410 (or end portion), a distal tip 406, and an intermediate or medial section 408. In certain embodiments, the distal tip 406 is rounded such that when the plunger 400 is manually deployed, it imparts a blunt force. The use of the blunt force within the bone marrow “cracks” or forms small pockets or cavities inside the cancellous bone, which allows fluid to gather around the main orifice around the needle insert 104. The plunger 400 also dears collective debris created during the insertion process from the needle insert longitudinal bore 132 in addition to around the needle insert 104 main orifice. In certain embodiments, the tip 406 is rounded with a radius of about 0.89 mm.

As similarly discussed above for the needle and the trocar, the length of the plunger 400 can vary based upon the length of the needle. For example, if the needle is 25 mm in length, then the plunger may be about 47.6 mm in length. Thus, in certain embodiments, the length of plunger insert 404 is about twice the length of the needle insert 104.

FIG. 5A shows an exploded view of the needle 100 and plunger 400. FIG. 5B shows an assembly 500 including the plunger 400 coupled to the needle 100. When the plunger insert 404 is fully inserted into the needle 100 such that the distal end of the plunger 400 extends beyond the needle tip 116, the grip 402 is then next to the grip 102. As will be explained below, this extension of the plunger 400 beyond the needle tip 116 forms the cavity in the marrow to aid in the extraction of the bone marrow.

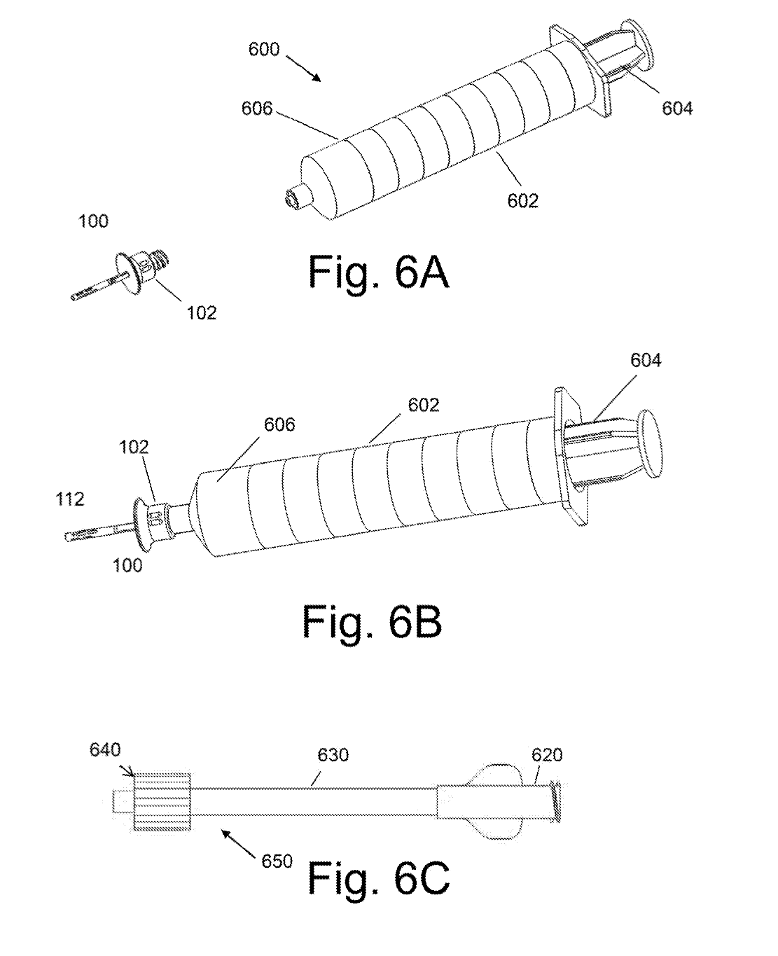

FIG. 6A is an exploded view of one embodiment of an aspiration assembly 600 showing the needle 102 and a suction device, such as syringe 602. FIG. 6B shows a side view of the aspiration assembly 600 in which the proximal end of the grip 102 of needle 100 is connected or coupled to the suction device 802, as shown in FIG. 6B. The suction device 602 can apply a negative pressure or suction force around the needle 100 to aspirate the bone marrow from the body via the fenestrations 112 and the general bore 132 of the needle.

Generally, the suction device 602 can comprise a surgical syringe as illustrated, however, any suitable suction device can be used with the needle to 100 to aspirate bone marrow. In the case of a surgical syringe 602, a syringe plunger 604 can be pulled upward and away from the body of the syringe 602 to create a vacuum, negative pressure or suction force with a space 606 of the syringe to cause the bone marrow to be withdrawn.

In an alternative embodiment, the suction device 602 is coupled to needle 100 via an additional tubing to add length if necessary. The tubing assembly 650 is shown in FIG. 6C having a female leur fitting end at the proximal end 620, an intermediate section of tubing 630 and a male leur fitting assembly at the distal end 640 in which the distal end 640 of tubing assembly 650 is coupled to needle 100 and the proximal end 620 of the tubing assembly 650 is coupled to the suction device 602.

### Operation

Referring now to FIGS. 1 through 8, the manner of using one embodiment of the present invention will now be described.

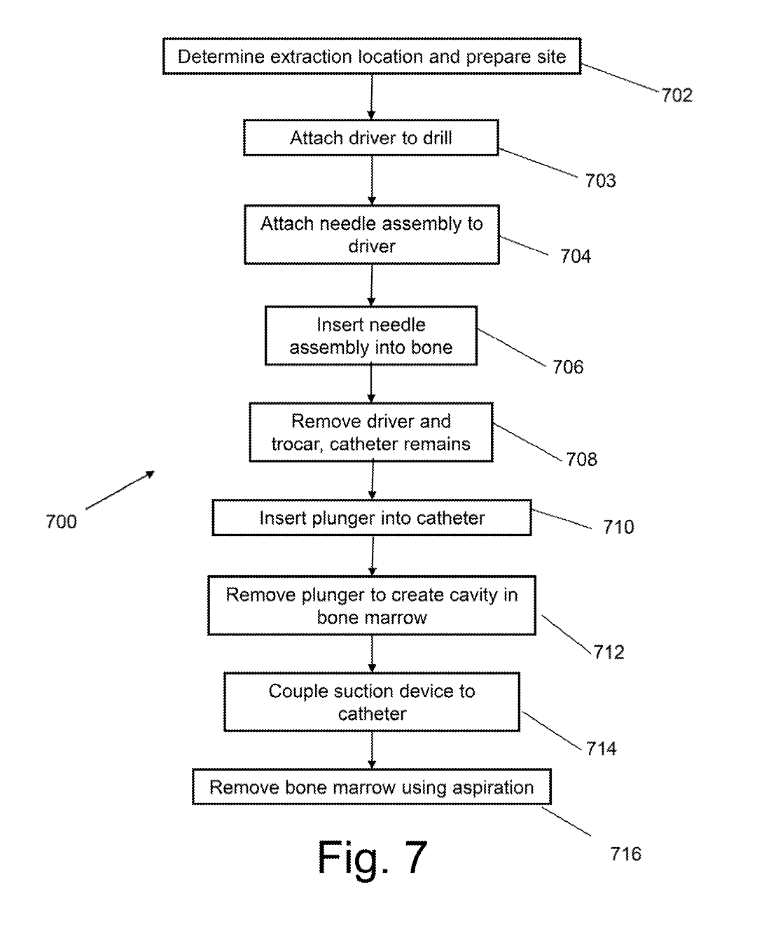

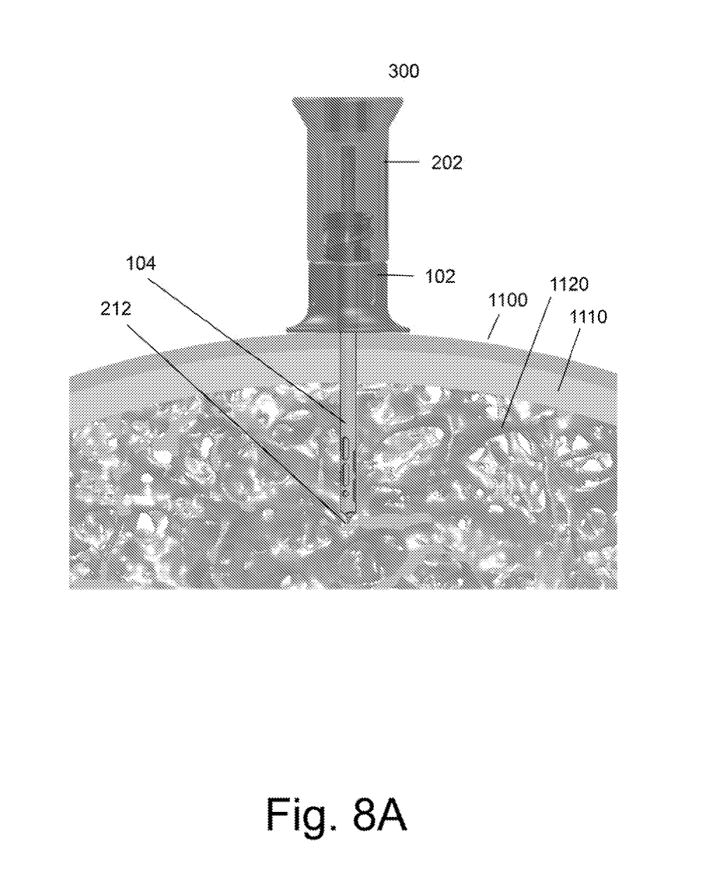

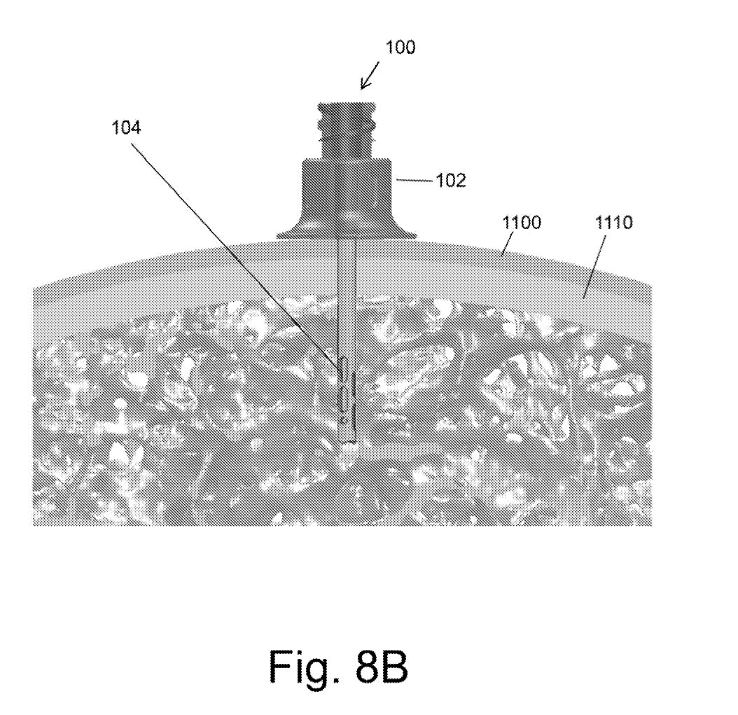

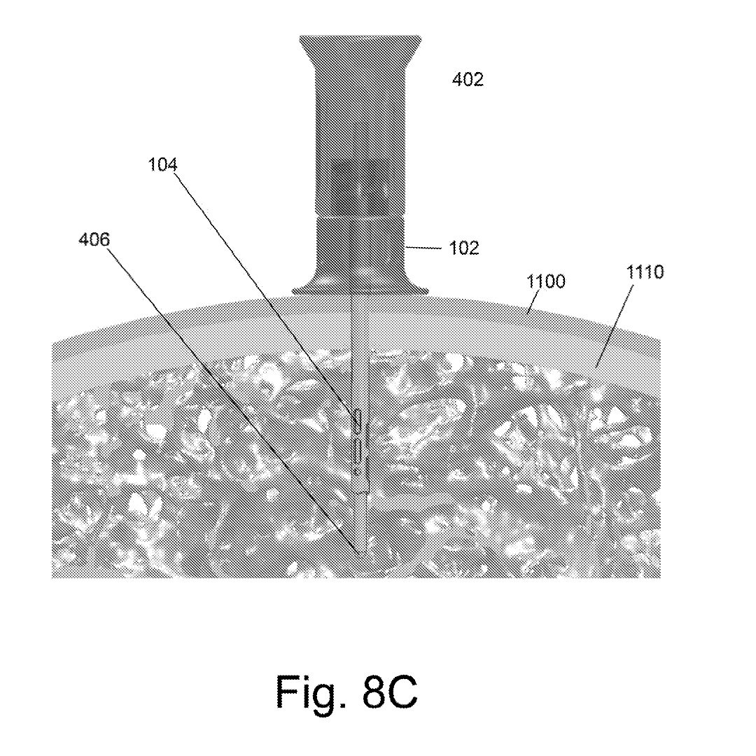

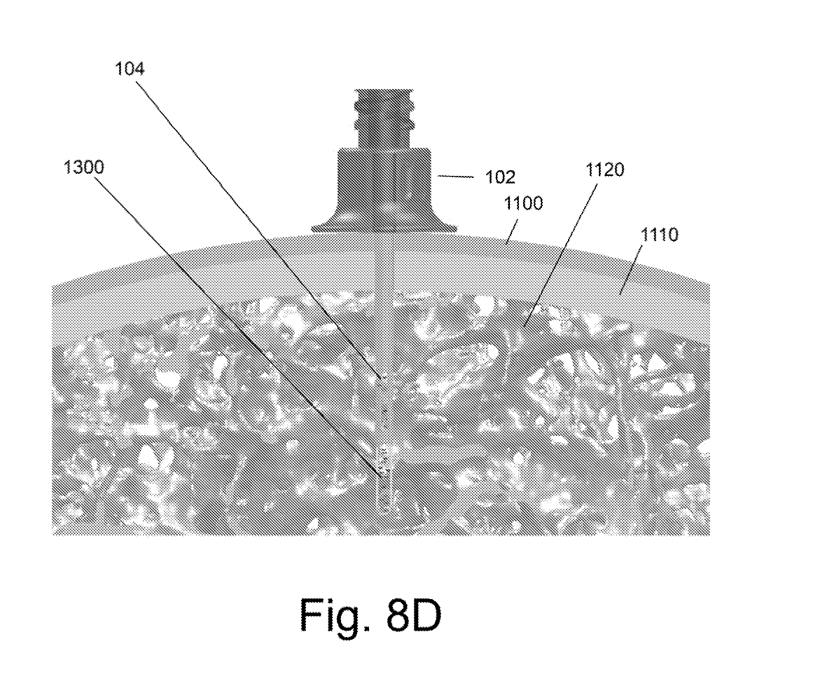

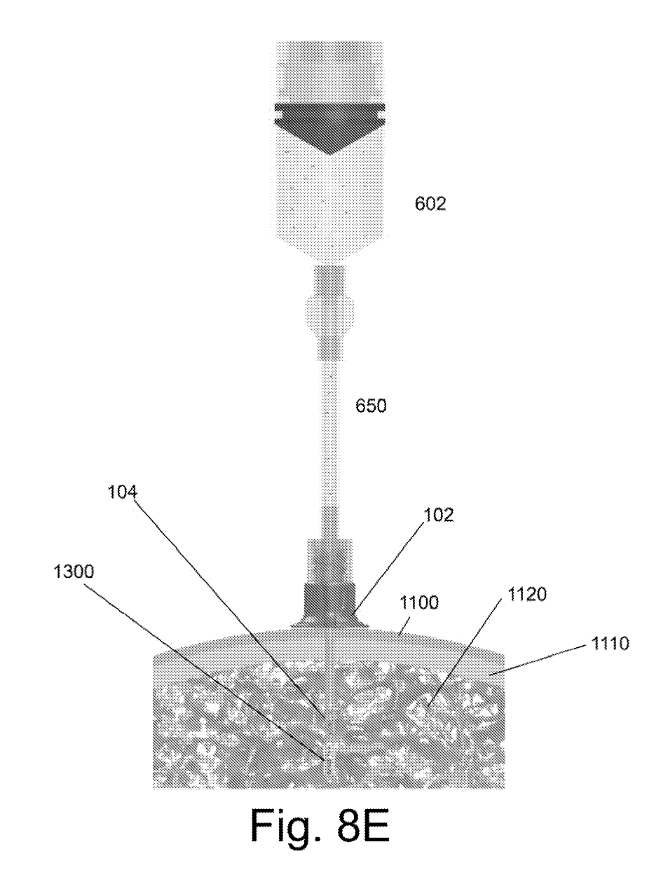

Turning to FIG. 7, there is an exemplary process to harvest bone marrow. Of course, this process is merely shown for the purpose of providing a general overview. One of skill in the art would be able to modify the process by adding steps and/or subtracting steps or performing them in a different order. FIGS. 8A-8E show an illustrative embodiment describing tine process illustrated by FIG. 7 and with a further illustration of an conceptual cross-section of bone marrow.

Referencing FIG. 7 and FIGS. 8A through 8E, the process starts at Step 702 where a medical worker determines the location of the extraction site and prepares the extraction site for the procedure. For example, an aseptic technique to prepare the skin at the site of extraction can be used and if further desired, a local anesthetic can be applied to the area. Next, at step 703 the driver 304 is coupled to a drill. Next, at step 704 the needle assembly 300 (needle and trocar) may be coupled to a driver. Having drawn and delineated landmarks to aid in insertion, the needle assembly 300 is inserted into the target extraction site using a handheld drill or other driver 304 (Step 706). When the drilling is complete, the driver may be disengaged as illustrated in FIG. 8A.

FIG. 8A provides an example of the needle assembly 300 inserted into the bone marrow 1120, through the bone 1110 and skin 1100. From the illustrated cross section it can be seen that the needle insert 104 is through the marrow and the distal tip 212 of the trocar is protruding through the needle insert 104. The needle grip 102 is seated directly against the skin and the trocar grip is seated on top of the needle grip 102.

FIG. 8B shows the removal of the trocar and the needle 100 remaining within the body (Step 708 of FIG. 7).

Next, in Step 710, the plunger 400 may be manually deployed or inserted until the plunger is fully seated on the proximal end of the needle grip 102 as illustrated in FIG. 8C which shows the distal tip of the plunger 406 protruding from the needle insert 104 and the plunger grip 402 is seated directly on the needle grip 102.

In Step 712, the plunger may he used to frack or crack the bone marrow and create a cavity. To achieve this fracking or cracking of the bone marrow, the plunger 400 is manually deployed past the tip of the needle 100 creating a fracture zone in the bone marrow around the tip of the needle 100. The plunger 400 will thus create a cavity, a cellular draw zone or fracture zone around the tip of the needle which increases the ability to draw bone marrow as well as decreasing the risk of platelet activation (by decreasing the amount of force necessary to withdraw the aspirate). This cavity created by plunger 400 allows fluid and cells to gather around the main orifice of the needle and to increase the flow during aspiration. FIG. 8D shows the removal of the plunger 400 and cavity 1300 represents the void that is left from the extension of the plunger into the bone marrow. This cavity 1300 allows fluid and cells to gather around the main orifice of the needle and to increase the flow during aspiration.

In step 714, a suction device 602 may be coupled to the needle grip 102. Then in step 716, the bone marrow may be drawn into the suction device via the negative pressure created by the suction device. FIG. 8E shows the suction device 602 coupled to the needle grip 102 via the alternative tubing 650 embodiment.

After the bone marrow is aspirated into the suction device, the extraction site is dressed as needed. In certain embodiments during the aspiration step 716, the needle may be rotated slightly to increase the area of marrow from which the needle is drawing or aspirating. Yet further, in certain other embodiments, steps 710-716 may be repeated as many times necessary to obtain the desired amount of bone marrow aspirate.

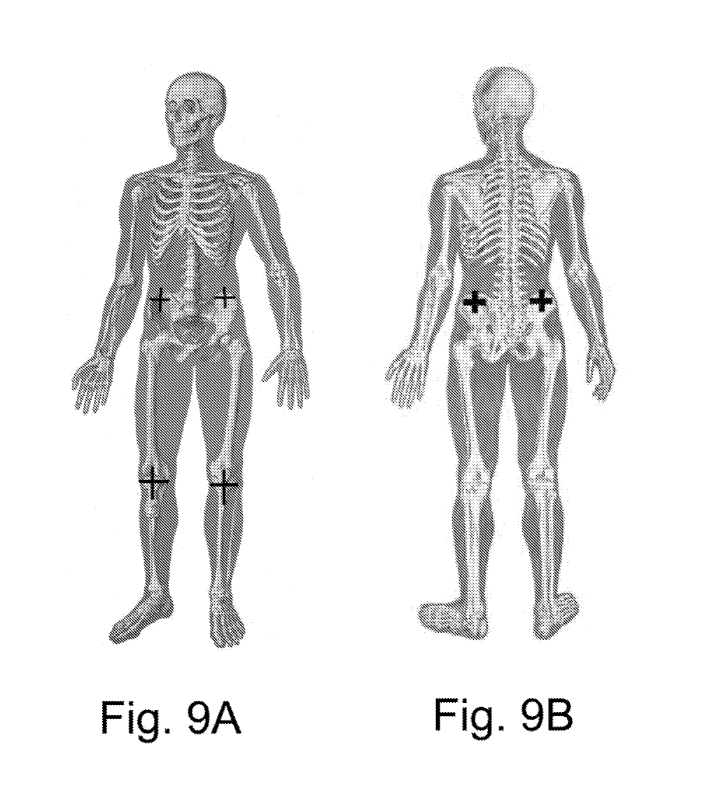

FIGS. 9A and 9B show the various possible target extraction sites. Thus, the site can be any bone having red marrow, for example, any cancellous or spongy or trabecular bone. Typically, for example, but not limited to the calcaneus bone (heel bone), tibia, the iliac crest, any spinal vertebra, etc. The spinal vertebra can include but are not limited to any cervical vertebra (C1, C2, C3, C4, C5, C6, C7), any thoracic vertebra (T1, T2, T3, T4, T5, T6, T7, T8, T9, T10, T11, T12), any lumbar vertebra (L1, L2, L3, L4, L5), or any sacral vertebra (S1, S2, S3, S4, S5). In certain embodiments, the spmai vertebra is selected from a lumbar vertebra, for example, L1, L2, L3, L4 and L5.

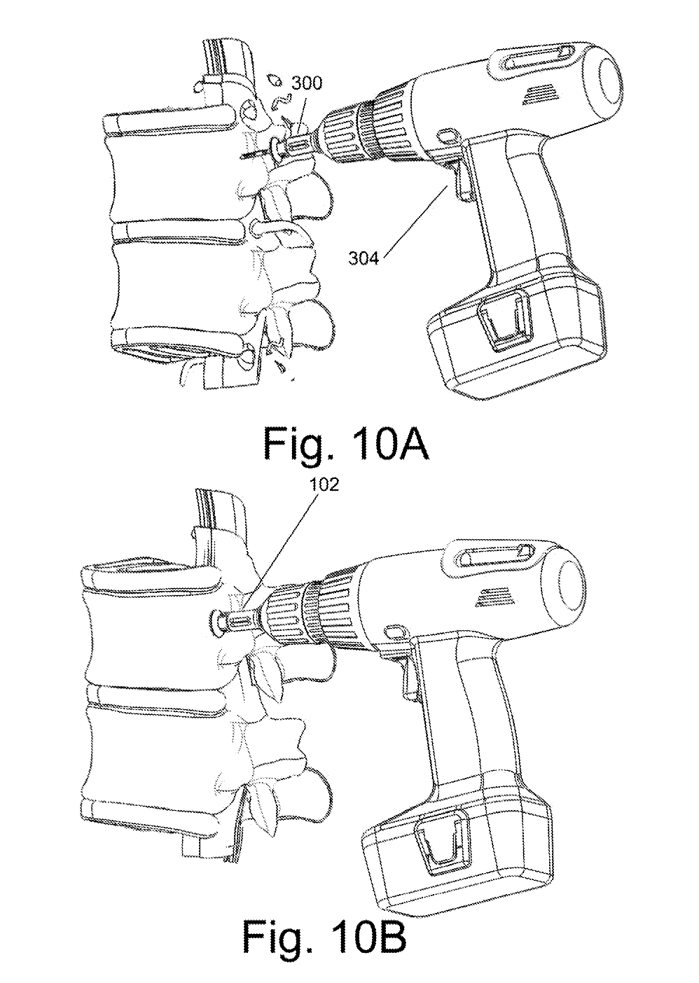

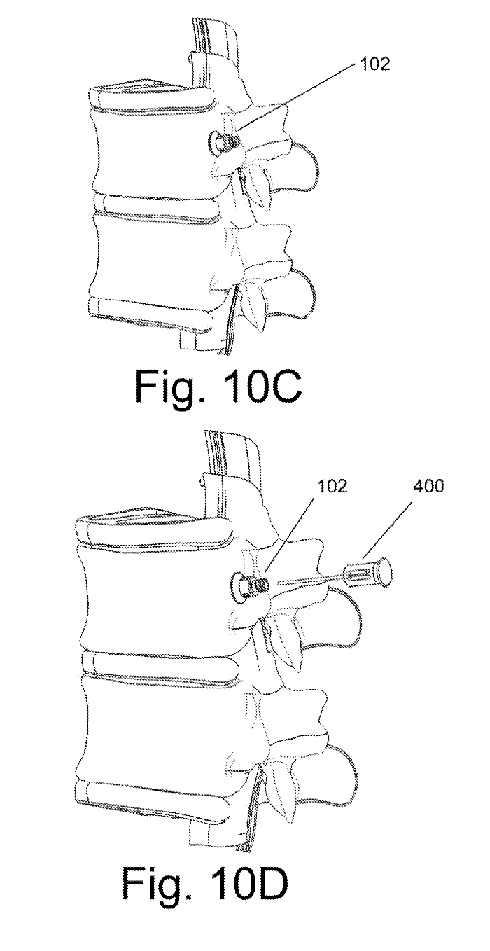

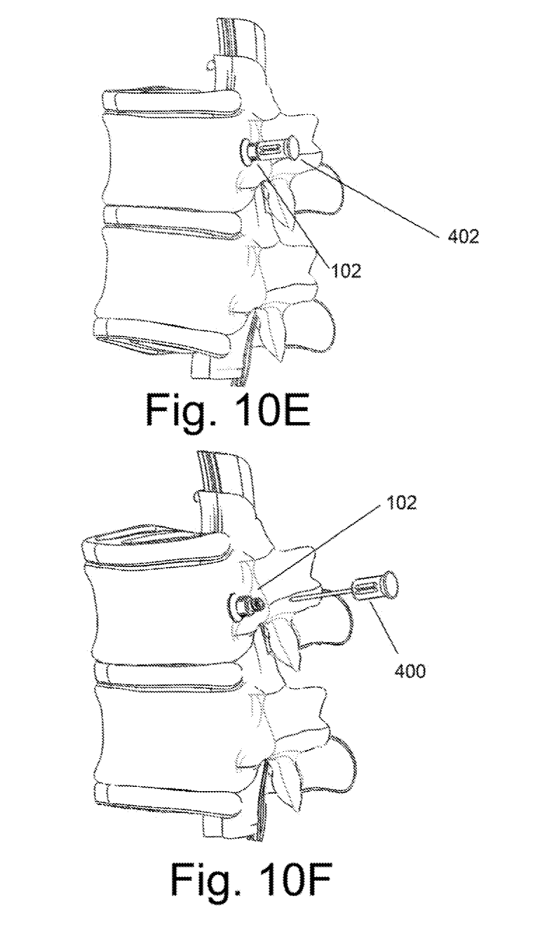

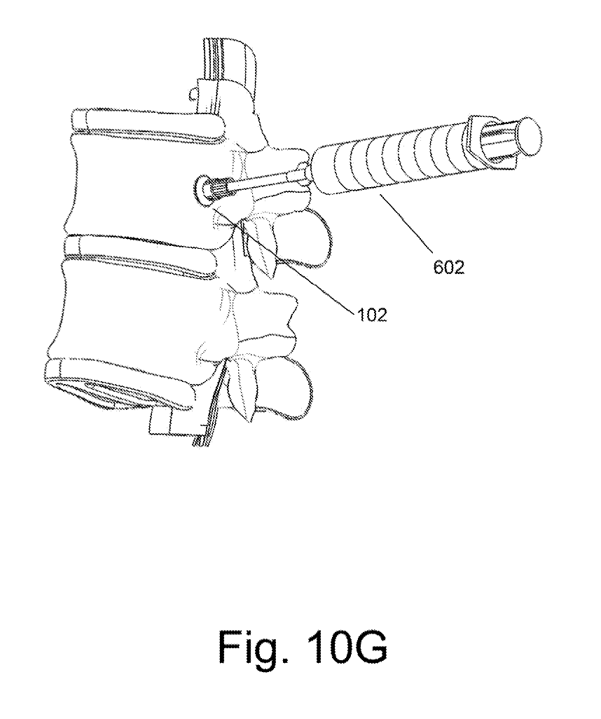

FIGS. 10A-10G illustrate an example of extraction of bone marrow from a vertebral body using the process or method as previously described in reference to FIG. 7. For the convenience of explanation and for clarity. FIGS. 10A-10G do not show the musculature or the skin associated with the vertebrae. FIG. 10A provides an example of inserting or drilling the needle assembly 300 into a spinous process using a handheld drill or driver 304, as described in Step 706 above. In an alternative embodiment, the physician can make a small incision directly through the skin instead of inserting the needle directly through the skin. In certain embodiments, the drilling is complete when the distal end of the grip 102 of needle assembly 300 is seated on the skin, as shown in FIG. 10B (skin is not directly shown). Next, the trocar 200 (not shown) is removed as shown FIG. 10C and a plunger 400 is manually deployed or inserted as shown in FIG. 10D (manually deployment is not shown) as shown in FIG. 10E, as further described previously in FIG. 7, Steps 708-712. Once plunger 400 has created fractured zones within the bone marrow (not shown), the plunger 400 is removed to create a cavity in the bone marrow, as shown in FIG. 10F. A suction device 602 is coupled to the proximal end of the needle grip 102 to aspirate the bone marrow as shown in FIG. 10G, as described in further detail in FIG. 7, Steps 714-716.

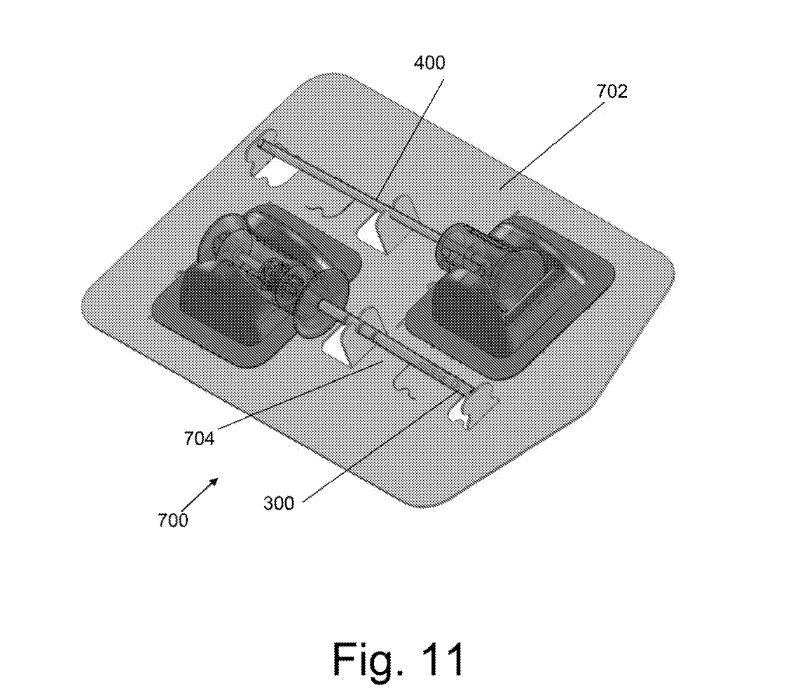

In certain embodiments, the needle assembly 300 and the plunger 400 can be packaged into a sterile package 702, as shown in FIG. 11 to form a sterile kit 700. As shown, package 702 contains the needle assembly 300 having a plastic protective sleeve 704 and the plunger 400. The package 702 is for single use only and may be sterilized using traditional radiation techniques such as cobalt₆₀ source gamma-irradiation in the range of 25-40 KGy to meet an SAL of 10⁻⁶, in a separate package (not shown), the adapter or drive mechanism 302 and the hand held drill 304 may be packaged for a one time purchase that is reusable after appropriate sterilization techniques standard to the medical profession.

It is envisaged that the needle assembly 300, as shown in FIGS. 1A-11 and described herein allows for a more efficient bone marrow extraction procedure. The efficiency of the bone marrow extraction or aspiration is increased by the design of needle 100, specifically, the addition of one or more fenestrations in the wall surface of the needle 100 allows for optimized flow characteristics upon extraction, thereby eliminating inefficient flow and cell damage. Furthermore, the fenestrations allow for added marrow volume to be extracted, as well as marrow from multiple locations without having to reposition or rotate the needle. In addition to the needle assembly 300, the system also employs a plunger, which is hand or manually deployed into the needle 100 after removal of the trocar 200 to form a pocket inside the cancellous bone. This pocket or cavity allows fluid to gather around the main orifice in the needle 100 aiding in extraction volume and cell viability. Thus, the kit and process described herein provides a method to harvest autologous mesenchymal stem cell from bone marrow aspirate that limits the clotting, limits the platelet cell activation, and maintains higher cell counts. The described process is generally tolerated well by the subject or patient, and it is believed to be much less painful, and less costly than any traditional bone marrow aspiration technique.

### Exemplary Therapy

Once the bone marrow aspirate has been collected, then the aspirate is separated into its various components using standard separation methods, such as centrifugation to collect a concentration of mesenchymal stem cells (MSC). Once the mesenchymal stem cells are collected, they are maintained in an aqueous physiological environment until they are injected or reintroduced into the subject. Once the cells have been separated and concentrated, they are prepared for injection into the patient to treat a variety of disorders. Thus, the therapeutic use of the MSC isolated using the described embodiments herein, may be injected into a predetermined site in an effective amount or effective treatment regimen to decrease, reduce, modulate or abrogate the disease and/or condition. Thus, a subject is administered a therapeutically effective amount of autologous MSC so that the subject has an improvement in the parameters relating to the disease and/condition. The improvement is any observable or measurable improvement. Thus, one of skill in the art realizes that a treatment may improve the patient condition, but may not be a complete cure of the disease.

Treatment regimens may vary as well. and often depend on the health and age of the patient. Obviously, certain types of disease will require more aggressive treatment while at the same time, certain patients cannot tolerate more taxing regimens. The clinician will be best suited to make such decisions based on the known subject's history.

For purposes of this invention, beneficial or desired clinical results include, but are not limited to, alleviation of symptoms, improvement of symptoms, dimishment of extent of disease, stabilized (i.e., not worsening) state of disease, delay or slowing of disease progression, amelioration or palliation of the disease state, and remission (whether partial or total), whether objective or subjective. The improvement is any observable or measurable improvement.

In one embodiment, the procedure may be used to augment the treatment of an osteoarthritic knee, and increase the body's chondrogenic potential in those osteochondral lesions of the joint surface by the use of the bone marrow aspirate concentrate or MSC. Bone marrow aspirate concentrate is unique in that it contains the autologous adult mesenchymal stem cells in a matrix of the patient's own growth factors derived from the concentration bone marrow aspirate.

Bone marrow aspirate concentrate/adult autologous mesenchymal stem cells may heal fractures and augment healing of nonunions by increasing the osteogenic potential of the human body. Such techniques using bone marrow aspirate concentrate/adult autologous mesenchymal stem cells may also be used to treat femoral neck fractures and help prevents subsequent collapse of the femoral head with avascular necrosis.

Other applications of the subject apparatus may be in tenogenesis and healing of the ligamentous attachments to bones after ligamentous sprayings of partial tears and tendon partial tears, in addition to tendon injuries and partial tears of the tendons or common flexor and common extensor insertion tears around the elbow. The subject apparatus may be used in the medial epicondyle around the elbow, lateral epicondyle, and in Achilles tendon injuries.

The abstract of the disclosure is provided for the sole reason of complying with the rules requiring an abstract, which will allow a searcher to quickly ascertain the subject matter of the technical disclosure of any patent issued from this disclosure. It is submitted with the understanding that it will not be used to interpret or limit the scope or meaning of the claims.

Any advantages and benefits described may not apply to all embodiments of the invention. When the word “means” is recited in a claim element, Applicant intends for the claim element to fall under 35 USC 112, paragraph 6. Often a label of one or more words precedes the word “means”. The word or words preceding the word “means” is a label intended to ease referencing of claims elements and is not intended to convey a structural limitation. Such means-plus-function claims are intended to cover not only the structures described herein for performing the function and their structural equivalents, but also equivalent structures. For example, although a nail and a screw have different structures, they are equivalent structures since they both perform the function of fastening. Claims that do not use the word means are not intended to fail under 35 USC 112, paragraph 6.

The foregoing description of the embodiments of the invention has been presented for the purposes of illustration and description. It is not intended to be exhaustive or to limit the invention to the precise form disclosed. Many combinations, modifications and variations are possible in light of the above teaching. For instance, in certain embodiments, each of the above described components and features may be individually or sequentially combined with other components or features and still be within the scope of the present invention. Undescribed embodiments which have interchanged components are still within the scope of the present invention. It is intended that the scope of the invention be limited not by this detailed description, but rather by the claims.

What is claimed is:

1. A method of a harvesting bone marrow from a body comprising:
a. providing a needle insert, wherein the needle insert has a longitudinal bore and one or more side fenestrations defined on a distal end of the needle insert,
b. inserting a trocar into a proximal end of the longitudinal bore,
c. positioning the trocar until a distal tip of the trocar extends longitudinally beyond a distal tip of the needle insert to create a needle assembly;
d. coupling the needle assembly to a drill;
e. drilling into a body such that the distal end of the trocar and the distal tip of the needle are positioned into a bone marrow of the body to a predetermined distance;
f. removing the trocar from the needle assembly white leaving a needle insert in the body;
g. repeatedly inserting a plunger through the needle such that a distal tip of the plunger extends beyond the distal tip of the needle to creating a fraction zone in the bone marrow;
h. removing the plunger from the needle insert;
i. coupling a suction device to the proximal end of the needle insert;
j. actuating the suction device to apply negative pressure around the distal tip of the needle insert; and
k. aspirating bone marrow through the one or more fenestrations of the needle into a portion of the suction device.

2. The method of claim 1 where the coupling the needle assembly to a drill further comprises coupling the needle assembly to a drill interface and coupling the drill interface to a drill.

3. The method of claim 1 further comprising locking the needle insert to the trocar.

4. A kit for use in harvesting bone marrow, comprising:
a. a needle having a distal end portion and a proximal end portion, said needle including:
i. a longitudinal bore running from said distal end portion to said proximal end portion,
ii. at least one fenestration defined through a side wall of said distal end portion and in communication with said longitudinal bore,
b. a trocar having a longitudinal shaft, wherein said longitudinal shaft is sized to be inserted into said needle and has a length such that a distal tip of said longitudinal shaft extends beyond said distal end portion of said needle when said longitudinal shaft is inserted into said needle; and
c. a plunger having a plunger longitudinal shaft, wherein said plunger longitudinal shaft is sized to be inserted into said longitudinal bore and has a plunger shaft length such that a portion of a distal end of said plunger longitudinal shaft extends beyond said distal end portion of said needle when said longitudinal shaft is inserted into said needle.

5. The kit of claim 4 wherein said needle includes four fenestrations defined through said distal end portion of said needle.

6. The kit of claim 4 wherein said needle includes eight fenestrations defined through said distal end portion of said needle.

7. The kit of claim 4 wherein said at feast one fenestration is comprised of at least two fenestrations of different shapes selected from the group consisting of oblong with radiused ends, circular, oval, polygonal or amorphous.

8. The kit of claim 7, wherein one of said at least two fenestrations is circular.

9. The kit of claim 7, wherein one of said at least two fenestrations is oblong with radiused ends.

10. The kit of claim 4 wherein said needle has a length of about 25 mm.

11. The kit of claim 4 wherein said needle has a length of about 15 mm.

12. The kit of claim 4 wherein said needle has a length of about 45 mm.
